(19) United States
(12) Patent Application Publication
(10) Pub. No.: US 2011/0124743 A1
(43) Pub. Date: May 26, 2011

(54) PALATABILITY ENHANCERS AND METHODS FOR ENHANCING PALATABILITY

(76) Inventor: Pascal Vanacker, Amiens (FR)

(21) Appl. No.: 12/737,004

(22) PCT Filed: May 21, 2009
(86) PCT No.: PCT/US09/03195

§ 371 Date: Jan. 27, 2011

Related U.S. Application Data
(60) Provisional application No. 61/130,942, filed on Jun. 3, 2008.

(51) Int. Cl. A61K 47/42; A61P 3/02; A23L 1/31; A23D 9/00; A23L 1/00

(52) U.S. Cl. 514/773; 426/641; 426/601; 426/289

(57) ABSTRACT

The invention provides palatability enhancers containing at least one animal digest and at least one surfactant and methods for using such palatability enhancers to enhance the palatability of comestible compositions.

### CROSS REFERENCE TO RELATED APPLICATIONS

This application is a national stage application under 35 U.S.C. §371 of PCT/US2009/003195 filed May 21, 2009, which claims priority to U.S. Provisional Application Ser. No. 61/130,942 filed Jun. 3, 2008, the disclosures of which are incorporated herein by reference.

### BACKGROUND OF THE INVENTION

1. Field of the Invention

This invention relates generally to palatability enhancers and particularly to palatability enhancers comprising at least one animal digest and at least one surfactant and methods for using such palatability enhancers to enhance the palatability of comestible compositions.

2. Description of Related Art

Generally, foods are generally designed to satisfy one or more nutritional requirements for an animal. In some cases, foods are designed to be a complete and nutritionally-balanced food that provides all known nutritional requirements for the animal. Although many foods are formulated primarily to satisfy various nutritional requirements, palatability of the foods must still be considered. Regardless of the actual nutritional content of a food, if an animal will not consume the food, or does not consume that food in sufficient quantities, the animal's nutritional needs may not be adequately met. Evidence suggests that having palatable foods provides benefits that go beyond an animal's purely physical or nutritional needs, e.g., reduction of stress hormones and a general feeling of health and wellness. Further, some conditions such as certain diseases and certain medications may suppress an animal's appetite. More palatable foods may be beneficial for such animals because they prevent or minimize poor nutrition for the animal if the animal consumes more of a more palatable food.

There are various ways of enhancing the palatability of food compositions. Addition of one or more ingredients that provide an appealing aroma or flavor can increase palatability of a food. Ingredients that affect food qualities such as aroma, flavor, texture, or mouthfeel, can all effect palatability. It is known that, for some foods, increasing sweetness or increasing fat or salt content can enhance palatability for some animals. Flavor enhancers, such as glutamic acid or its salts and various nucleotide or ribonucleotide compounds are known in the art to be palatability enhancers. Flavor enhancers that enhance the existing flavor in a food are commonly used in savory foods. It may not be desirable for such flavor enhancers to add any flavor of their own. In contrast, palatability enhancers in some cases, not only may add flavor or aroma or other qualities that effect palatability, but may be specifically chosen for those attributes.

There are many known methods and compositions useful for enhancing palatability. For example, US20080085350A1 discloses a method for producing a palatability enhancer for a companion animal food product, wherein the method includes combining at least one aroma chemical selected from 2-methylfuran, 2-methylpyrrole, 2-methyl-thiophene and dimethyl disulfide with an animal or vegetarian digest composition. US20050276881A1 discloses a catnip palatability enhancer comprising nepetalactone in form of catnip, catnip oil, matatabi, extract of matatabi, nepetalactone, and/or active analog of nepetalactone. US20010002272A1 discloses enhancing the palatability of extruded pet foods using tetrasodium pyrophosphate. US20050037108A1 discloses palatability enhancers containing tetrapotassium pyrophosphate and additional ingredients selected from animal by-products, microbial proteins, dairy by-products, amino acids. U.S. Pat. No. 7,329,426 discloses the preparation of a palatability enhancer for animal food by cooking a liquefied mixture of triglyceride molecules from plant or animal source, mixed with at least one non-cellular donor compound as donor of sulfur or nitrogen.

Even though there are many known methods and compositions useful for enhancing palatability, there is always a need for new palatability enhancers that make foods and other comestible compositions more palatable.

### SUMMARY OF THE INVENTION

It is, therefore, an object of the invention to provide palatability enhancers useful for enhancing the palatability of comestible compositions such as food compositions, dietary supplements, medicaments, or other edible materials.

It is another object of the invention to provide comestible compositions such as foods, dietary supplements, medicaments, or other edible materials having enhanced palatability.

It is a further object of the invention to provide methods for enhancing the palatability of comestible compositions.

It is another object of the invention to provide methods for making palatability enhancers useful for enhancing the palatability of comestible compositions.

One or more of these and other objects are achieved using novel palatability enhancers that enhance the palatability of comestible compositions such as foods, dietary supplements, medicaments, or other edible materials. The palatability enhancers comprise at least one animal digest and at least one surfactant, preferably a non-ionic surfactant, in an amount effective for enhancing the palatability of comestible compositions.

Other and further objects, features, and advantages of the invention will be readily apparent to those skilled in the art.

### DETAILED DESCRIPTION OF THE INVENTION

### Definitions

The following abbreviations may be used herein: AAFCO, Association Of American Feed Control Officials, Inc.; HLB, hydrophilic-lipophilic balance; MEA, monoethanolamine; DEA, diethanolamine; and TEA, triethanolamine.

The term “animal” is used in a general sense and means a human or other animal that may choose a comestible composition based upon its palatability, including avian, bovine, canine, equine, feline, hicrine, lupine, murine, ovine, and porcine animals.

The term “companion animal” means any domesticated animal, and includes, without limitation, cats, dogs, rabbits, guinea pigs, ferrets, hamsters, mice, gerbils, horses, cows, goats, sheep, donkeys, pigs, and the like. Certain embodiments in which human animals are expressly excluded, or clearly excluded by the context, may be preferred herein. In certain embodiments, companion animals are preferred, for example, dogs and cats. Some companion animals receive the same dietary food, such as a complete and nutritionally-balanced food, e.g., a kibble, for prolonged periods. The palatability enhancers of the invention are particularly useful for enhancing the palatability of such foods, and thereby, benefiting the nutrition and health status of the animal.

The terms “palatable” or “palatability” refer to a quality of a comestible composition such as a food, food supplement, food additive, dietary supplement, medicament, or the like, that makes it appealing or pleasing to one or more of an animal's senses, particularly the senses of taste and smell. Accordingly, palatability is determined subjectively. As used herein, whenever an animal shows a preference, for example, for one of two or more foods, the preferred food is more “palatable,” and has greater “palatability.” For companion animals and other non-human animals, the relative palatability of one food compared to one or more other foods can be determined, for example, in side-by-side, free-choice comparisons, e.g., by relative consumption of the foods, or other appropriate measures of preference indicative of palatability.

The skilled artisan will appreciate that various aspects or phases of “palatability” can be considered both independently and interdependently. For example, “initial appeal,” “continued consumption palatability,” and “repeated presentation palatability” can all be considered. “Initial appeal” is an aspect of palatability that induces an animal to initially taste or try a food, dietary supplement, or medicament. “Continued consumption palatability” is an aspect of palatability that induces an animal to continue consuming a product that has been initially only tasted or tried. “Repeated presentation palatability” or “repeated feeding palatability” is an aspect of palatability evident when a food composition, dietary supplement, or medicament, which has previously been both tasted and consumed, is presented repeatedly to the animal for consumption over time. For example, a complete and nutritionally-balanced food composition that is fed daily to an animal will hopefully provide palatability for each repeated presentation of feeding, such that the animal continues to consume adequate quantities of the food.

The term “palatability enhancer” means any compound, composition, formulation, or other material useful for enhancing the palatability of a comestible composition such as a food composition, supplement, medicament, or the like. Palatability enhancers enhance palatability at any one or more of the aspects of palatability. Thus, such palatability enhancers may contribute to initial appeal, continued consumption, or repeated presentation aspects of palatability, or any combination thereof. Examples of palatability enhancers include fats (e.g., tallow), flavors, aromas, extracts, digests, and the like.

The term “surfactants” means compounds that are surface active. Surfactant molecules typically have a hydrophilic portion (e.g., one or more head groups) and a hydrophobic (or lipophilic) portion (e.g., one or more tails). Surfactants can be classified in various ways, for example they may be classified according their hydrophilic-lipophilic balance (HLB). Based on the presence or absence of formally-charged in the head group(s), surfactants can be classified as non-ionic, ionic, or zwitterionic. The heads of non-ionic surfactants have no charged groups. Ionic surfactants feature at least one charged group in their heads. If the group is negatively-charged, the surfactant is an anionic surfactant. If the charge on the head group(s) is positive, the surfactant is a cationic surfactant. Surfactants that contain a head with two oppositely-charged groups are known as zwitterionic surfactants.

The term “non-ionic surfactant” means any of a group of surfactants comprising one or more noncharged, hydrophilic heads, and one or more generally polar tails. A functional application of non-ionic surfactants is to reduce surface tension at interfaces between water and oil, for example in complex systems. The non-ionic surfactant is preferably capable of forming an emulsion of a palatability enhancing composition. Common types of non-ionic surfactants include those containing amide linkages, such as cocamide MEA, cocamide DEA, and cocamide TEA, and those containing ester groups, such as ethyleneglycol monostearate, ethyleneglycol distearate. Some examples of non-ionic surfactants include poloxamers (e.g., Pluronics), polyoxyethylene alkyl ethers (e.g., Brij), and polyoxyethylene sorbitan fatty acid esters (e.g., Tween). The surfactant preferably has a hydrophilic-lipophilic balance (HLB) greater than 10, more preferably greater than 12 and most preferably greater than 14.

In some embodiments, the non-ionic surfactants useful herein comprise either a derivatized edible or food-compatible oil or fatty acid molecule, or an esterified or ethyoxylated food carbohydrate. Additional examples of non-ionic surfactants that may be useful with compositions provided herein include alkyl poly(ethylene oxide) compounds, copolymers of poly(ethylene oxide) and poly(propylene oxide) (e.g., Poloxamers or Poloxamines); alkyl glucosides or polyglucosides, such as octyl glucoside and decyl maltoside; fatty alcohols, such as cetyl alcohol and oleyl alcohol, Tween 80 (polyoxyethylenesorbitan monooleate); cocamide ethanolamines including cocamide MEA, cocamide DEA, and cocamide TEA. Suitable surfactants also include polyoxyethylene derivatives of edible oils or vegetable oils or hydrogenated edible oils or hydrogenated vegetable oils, polyethoxylated castor oils or polyethoxylated hydrogenated castor oil, polyoxyethylene-sorbitan-fatty acid esters, polyoxyethylene castor oil derivatives and the like. Also useful herein are surfactants comprising lecithin derivatives, propylene glycol fatty acid esters, glyceryl fatty acid esters, polyglyceryl fatty acid esters, polyoxyethylene glyceryl fatty acid esters, sorbitan fatty acid esters, polyoxyethylene sorbitan fatty acid esters, polyoxyethylene sorbitol fatty acid esters, polyethylene glycol fatty acid esters, polyoxyethylene castor oils or hydrogenated castor oils, polyoxyethylene alkyl ethers, polyoxyethylene phytosterols, polyoxyethylene polyoxypropylene alkyl ethers, or polyoxyethylene lanolins/lanolin alcohols/beeswax derivatives.

Commercial non-ionic surfactants useful in pharmaceutical and food applications may also be useful herein. Examples include TWEEN 20 (Polyoxyethylene sorbitan monolaurate), TWEEN 40 (Polyoxyethylene sorbitan monopalmitate), TWEEN 60 (Polyoxyethylene sorbitan monostearate), TWEEN 80 (Polyoxyethylene sorbitan monooleate), NIKKOL HCO30 (PEG-30 hydrogenated castor oil), NIKKOL HCO40 (PEG-40 hydrogenated castor oil), NIKKOL HCO50 (PEG-50 hydrogenated castor oil), NIKKOL HCO60 (PEG-60 hydrogenated castor oil), CREMOPHORE RH40 (Polyoxyethylene 40 castor oil), CREMOPHORE RH60 (Polyoxyethylene 60 castor oil), and CREMOPHORE EL35 (Polyoxyethylene 35 castor oil).

The term “animal digest” means material which results from chemical and/or enzymatic hydrolysis of clean, undecomposed animal tissue. In certain embodiments, “animal digest” as used herein, is fully consistent with the definition of animal digest promulgated by the Association Of American Feed Control Officials, Inc. (AAFCO). Animal digest is preferably derived from animal tissues, including cold-blooded marine animals, excluding hair, horns, teeth, hooves, and feathers. The skilled artisan will appreciate that while such tissues are not preferred, trace amounts might be found unavoidably even under good manufacturing practices. Also not included are visceral contents or foreign or fecal matter, although trace contaminant amounts are sometimes present. When an animal digest is dried, it may be referred to as “dried animal digest.” Animal digests in accordance herewith are suitable for use in food or feed compositions. Specifically included are (1) Digest of Beef (or Poultry, Pork, Lamb, Fish, etc): material from beef (poultry, pork, etc.) which results from chemical and/or enzymatic hydrolysis of clean and undecomposed tissue; (2) Digest of Beef (or Pork, Lamb, etc) By-Products: material from beef (poultry, pork, etc.) which results from chemical and/or enzymatic hydrolysis of clean and undecomposed tissue from non-rendered clean parts from cattle (pigs, lambs, fish, etc), other than meat, for example lungs, spleen, kidneys, brain, livers, blood, bone, partially-defatted low-temperature fatty tissue, and stomachs and intestines, freed of their contents; and (3) Digest of Poultry By-Products: material which results from chemical and/or enzymatic hydrolysis of clean and undecomposed tissue from non-rendered clean parts of carcasses of slaughtered poultry such as heads, feet, and viscera. As used herein “poultry” encompasses any species or kind of bird, preferably chicken, turkey, duck, or other food species.

The term “effective amount” means an amount of a compound, material, composition, medicament, surfactant, or other material that is effective to achieve a particular desired result. Such results include, but are not limited to, one or more of the following: (a) enhancing palatability; (b) inducing an animal to consume more of a particular comestible composition than it otherwise would, in either a single feeding, or over the course of multiple feedings; or (c) inducing an animal to consume a medicament or a food or dietary supplement that the animal might not otherwise voluntarily consume.

The term “food” or “food product” or “food composition” means a product or composition that is intended for ingestion by an animal, including a human, and provides at least one nutrient to the animal. The term “food” includes any food, feed, snack, food supplement, treat, meal substitute, or meal replacement, whether intended for a human or another animal. “Food” encompasses such products in any form, solids, liquids, gels, or mixtures or combinations thereof. Thus, beverages of any type are clearly encompassed within the term “food.”

“Animal food” includes food or feed intended for any domesticated or wild species. In preferred embodiments, a food for an animal represents a nutritionally-complete food composition, e.g., a pelleted, extruded, or dry food.

The term “pet food” means a composition intended for consumption by an animal, preferably by a companion animal. Nutritionally-balanced pet foods are widely known and used in the art.

A “nutritionally-complete” “nutritionally-balanced” or “complete and nutritionally-balanced food” is one that contains all known required nutrients for the intended recipient or consumer of the food, in appropriate amounts and proportions, based, for example, on recommendations of recognized or competent authorities in the field of companion animal nutrition. Such foods are therefore capable of serving as a sole source of dietary intake to maintain life or promote production, without the addition of supplemental nutritional sources. The terms include any food, feed, snack, food supplement, treat, meal substitute, or meal replacement, whether intended for a human or another animal, in any form, including solids, liquids, gels and the like. Such foods, when intended for companion animals, are frequently in the form of extruded pet foods, such as kibble-type foods for dogs and/or cats.

The term “dietary supplement” means a product that is intended to be ingested in addition to the normal animal diet. Dietary supplements may be in any form, e.g., solid, liquid, gel, tablets, capsules, powder, and the like. Preferably they are provided in convenient dosage forms. In some embodiments they are provided in bulk consumer packages such as bulk powders, liquids, gels, or oils. In other embodiments, supplements are provided in bulk quantities to be included in other food items such as snacks, treats, supplement bars, beverages and the like. Palatability enhancing compositions can be used to improve palatability of dietary supplements in the same manner as they are used to improve palatability of foods.

The term “in conjunction” means that a composition for enhancing the palatability of a comestible composition such as a food composition, dietary supplement, or medicament (palatability enhancer), and a food composition or medicament whose palatability is to be enhanced, described herein are administered to an animal (1) together in a food composition, dietary supplement, or medicament, or (2) separately, at the same or different frequency, using the same or different administration routes, at about the same time, or periodically. “Periodically” means that the agent is administered on a dosage schedule acceptable for that specific enhancing agent and that the food, dietary supplement, or medicament, is provided to an animal routinely as appropriate for the particular animal. “About the same time” generally means that the food, dietary supplement, or medicament, and the enhancing agent are administered at the same time or within about 72 hours of each other. “In conjunction” specifically includes administration schemes wherein an enhancing agent is administered for a predetermined, prescribed, or desired period, and the compositions disclosed herein are administered within a defined window of time before, during, or after providing the food, dietary supplement, or medicament whose palatability is to be enhanced, the window being between about 0-240 minutes before the start of, and after the completion of, e.g., the animal's normal feeding time, supplement time, or medicament administration time.

The term “single package” means that the components of a kit are physically associated in or with one or more containers and considered a unit for manufacture, distribution, sale, or use. Containers include, but are not limited to, bags, boxes, cartons, bottles, packages of any type or design or material, over-wrap, shrink-wrap, stapled or otherwise affixed components, or combinations thereof. A single package may be containers of individual palatability enhancers and food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain, for example, instructions on how to use the kit, or safety or technical information about one or more components of a kit. Examples of information that can be provided as part of a virtual kit include instructions for use; safety information such as material safety data sheets; poison control information; information on potential adverse reactions; clinical study results; dietary information such as food composition or caloric composition; general information on improving palatability in the diet, or increasing appetite in an animal in need thereof; health consequences stemming from a decrease in nutrient intake, or from inadequate nutrient intake; or general information on nutrition or providing optimal nutrition; self-help relating to nutrition and appetite; caregiver information for those caring for animals with nutritional challenges, and diseases that result in decreased body weight, wasting, or the like, or other loss of appetite challenges; improving acceptance of orally-administered dietary supplements or medicaments, and use, benefits, and potential side-effects or counter-indications, if any, for the compositions described herein, e.g., palatability enhancers.

All percentages expressed herein are by weight of the composition on a dry matter basis unless specifically stated otherwise. The skilled artisan will appreciate that the term “dry matter basis” means that an ingredient's concentration or percentage in a composition is measured after the free water has been removed, or determined on the basis of the weight of the composition after any the weight of any free moisture in the composition has been subtracted.

As used throughout, ranges herein are stated in shorthand, so as to avoid having to set out at length and describe each and every value within the range. Any appropriate value within the range can be selected, where appropriate, as the upper value, lower value, or, the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “a surfactant”, “a method”, or “a food” includes a plurality of such “surfactants”, “methods”, or “foods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Where used herein the term “examples,” particularly when followed by a listing of terms is merely exemplary and illustrative, and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### THE INVENTION

In one aspect, the invention provides palatability enhancers useful for enhancing the palatability of comestible compositions such as food compositions, dietary supplements, medicaments, or other edible materials. The palatability enhancers comprise at least one animal digest and at least one surfactant in an amount effective for enhancing the palatability of a comestible composition. The inventions is based upon the surprising discovery that adding a surfactant to an animal digest increases the palatability of the animal digest and comestible compositions comprising the animal digest.

In general, compositions, compounds, or other materials that enhance palatability are referred to herein and in the art as “palatability enhancers,” “palatability enhancing agents,” “palatability agents,” and the like. Palatability enhancers differ from “flavor enhancers” in that the latter generally are used only with savory (or umami) foods, and preferably do not add any flavor of their own. The palatability enhancers of the invention enhance palatability by affecting any quality of a comestible composition that contributes to palatability. In various embodiments, the palatability enhancers enhance palatability by enhancing the aroma, flavor, texture, mouthfeel, satiety, or other aspect of the palatability of a comestible composition. In some embodiments, the palatability enhancers at least partially mask a flavor, aroma, or other quality that would otherwise contribute to a low palatability of a comestible composition.

The surfactants can be any surfactant known to skilled artisans that is compatible with animal digests and that enhances palatability when combined with the animal digests. In preferred embodiments, the palatability enhancers comprise at least one non-ionic surfactant. The non-ionic surfactants can be any non-ionic surfactant. Preferably, the non-ionic surfactants comprise an alkyl poly(ethylene oxide); a copolymer of poly(ethylene oxide) and poly(propylene oxide); an alkyl glucoside or polyglucoside; a fatty alcohol; a cocamide mono-, di-, or tri-ethanolamine, a lecithin derivative, a propylene glycol fatty acid ester, a glyceryl fatty acid ester, a polyglyceryl fatty acid ester, a polyoxyethylene glyceryl fatty acid ester, a sorbitan fatty acid ester, a polyoxyethylene sorbitan fatty acid ester, a polyoxyethylene sorbitol fatty acid ester, a polyethylene glycol fatty acid ester, a polyoxyethylene castor oil or hydrogenated castor oil, a polyoxyethylene alkyl ether, a polyoxyethylene phytosterol, a polyoxyethylene polyoxypropylene alkyl ether, or a polyoxyethylene lanolin, lanolin alcohol, or beeswax derivative.

Palatability enhancers comprising non-ionic polyoxyethylene sorbitan fatty acid esters are surprisingly effective for increasing or enhancing palatability. As shown in the examples, animals consumed the foods with added palatability enhancers comprising non-ionic polyoxyethylene sorbitan fatty acid esters with a preference of over 3:1 as compared to controls that did not include the palatability enhancers. Polyoxyethylene sorbitan fatty acid esters are commercially available as TWEEN products, including for example, TWEEN 20, TWEEN 40, TWEEN 60, and TWEEN 80, which are respectively the monolaurate, monopalmitate, monostearate, and monooleate esters.

In preferred embodiments, the non-ionic surfactant is a polyoxyethylene sorbitan fatty acid monooleate or a TWEEN 80 formulation.

The animal digest can be any animal digest known to skilled artisans that is compatible with surfactants and that enhances palatability when combined with the surfactants. In preferred embodiments, the animal digests comprise beef, pork, poultry, lamb, fish, or any combination thereof. Some embodiments include animals digests made substantially from nonrendered parts of animals, for example viscera, free from any contents thereof. In one embodiment, the animal digest comprises poultry and/or pork digest. Because different animals may find different animal digests more palatable than others, the skilled artisan will appreciate that different animal digest may be preferred for different animals. Further, as the skilled artisan will appreciate, by varying the palatability enhancer for example by using different animal digests, or varying the amounts, sources, or composition of the animal digest, more variety in the diet can be perceived by the animal, even where the base food composition or formulation remains relatively unchanged.

The palatability enhancers generally comprise from about 0.05% to about 10% surfactant and from about 90% to about 99.95% animal digest. In various embodiments, the surfactant comprises from about 0.05% to 5% of the palatability enhancer and from about 95% to about 99.95% animal digest, preferably from about 0.1% to 3% surfactant and from about 97% to about 99.9% animal digest, and more preferably from about 0.25% to about 1% surfactant and from about 99% to about 99.75% animal digest.

In various embodiments, the palatability enhancer further comprises one or more additional components that enhance palatability of a comestible composition such as a food, dietary supplement, or medicament. Examples of such components include food-compatible acids, acidifiers, or salts thereof, salt (e.g., NaCl, or KCl), sweetening agents, edible fats, flavor-enhancing amino acids (e.g., glutamic acid) or derivatives or salts thereof, flavor-enhancing nucleotides or ribonucleotides or derivatives or salts thereof. Additional components that can enhance palatability include components that alter other qualities of the food, dietary supplement, or medicament, for example, texture, consistency, mouthfeel, after-taste, melting point, and others.

Any food-compatible or food-grade acid or salts (especially sodium, calcium, or potassium salts) or derivatives thereof can be used in conjunction with the palatability enhancing compositions provided herein. Acids including organic acids such as various fatty acids, citric acid cycle acids, sugar acids, and keto acids may all be added to palatability enhancing compositions. In various embodiments, the compositions comprises one or more organic acids such as formic acid, acetic acid, citric acid, propionic acid, lactic acid, malic acid, fumaric acid, capric acid, caproic acid, butyric acid, hexamic acid, tartaric acid, gluconic acid, glutamic acid, glucuronic acid, guanylic acid, inosinic acid; kojic acid, and itaconic acid. Also useful are phosphoric acid, and salts and derivatives thereof, as well sodium bisulfate, pyrophosphoric acid, acid tripolyphosphate, and acid hexapolyphosphate.

In another aspect, the invention provides comestible compositions such as food compositions, dietary supplements, medicaments, or other edible materials having enhanced palatability. The comestible compositions comprise one or more ingredients suitable for consumption by an animal and at least one palatability enhancer comprising at least one animal digest and at least one surfactant in an amount effective for enhancing the palatability of the comestible composition. The animal digest and surfactant in the palatability enhancer are present in amounts of from about 0.05% to about 10% surfactant and from about 90% to about 99.95% animal digest. In various embodiments, the surfactant comprises from about 0.05% to 5% of the palatability enhancer, preferably from about 0.1% to 3% and more preferably from about 0.25% to about 1%, with the remainder being animal digest and other optional ingredients. The comestible composition, depending on the particular composition, comprises from about 0.001% to about 90% palatability enhancer, preferably from about 0.05% to 50%, more preferably from about 0.1% to 30%, and most preferably from about 1% to about 10%. For example, in a food composition, the comestible composition may comprise about 10% palatability enhancer whereas in a dietary supplement, particularly one that is not very palatable, the comestible composition may comprise 90% palatability enhancer.

In another aspect, the invention provides a food composition comprising one or more edible food ingredients and a palatability enhancer comprising at least one animal digest and at least one surfactant in an amount effective for enhancing the palatability of the food composition. Preferably, the surfactant is a non-ionic surfactant.

In various embodiments, the food composition comprises from about 0.001% to about 10% palatability enhancer. In one embodiment, the composition comprises from about 0.05% to 5% of the palatability enhancer, more preferably from about 0.1% to 3%, and most preferably from about 0.25% to about 1%. When the palatability enhancer is applied as a surface application on the food composition, for example, as a coating, concentration of the palatability enhancer required may be lower to accomplish the same degree of palatability enhancement.

In a preferred embodiment of the food composition, the palatability enhancer comprises a polyoxyethylene sorbitan fatty acid ester. In one embodiment, the palatability enhancer is topically applied.

In another aspect, the invention provides an extruded food composition suitable for consumption by a companion animal. The extruded food composition comprises a palatability enhancer comprising at least one animal digest and at least one surfactant. Preferably, the composition comprises less than 10% moisture (or water) content, for example 10, 9, 8, 7, 6 or even 5% moisture, or less. In other embodiments, the moisture content of the extruded food is less than 5, 4, or 3%, while in yet other embodiments the composition is less than 2.5% moisture, or even 2 or 1% or less. Such compositions are shelf-stable and commercially-dry. Preferably, the extruded food compositions resist bacterial and mold degradation because the moisture content and water activity is too low to support growth of such organisms. In one embodiment, the extruded composition is in the form of a kibble, particularly a kibble suitable for consumption by a canine or feline such as a dog or a cat. Preferably, the surfactant is a non-ionic surfactant.

The palatability enhancer is adapted to appeal to at least one sense of a companion animal for which it is intended by adjusting the content animal digest to the preference of the targeted companion animal. For example, the animal digest targeted for dogs may preferentially comprise beef, pork, poultry, lamb, or combinations thereof while animal digest targeted for cats may preferentially comprise poultry, fish, or combinations thereof.

In another aspect, the invention provides a dietary supplement or medicament comprising at least one active ingredient, e.g., a nutrient or active medicinal ingredient, and a palatability enhancing composition comprising at least one animal digest and at least one surfactant. Presently preferred are dietary formulations or medicaments that are intended for veterinary applications. Some dietary supplements and medicaments are difficult to administer because animals, such as companion animals, do not like the dietary supplement or medicament and will not readily ingest it. For supplements or medicaments that are critical to the animal's health, failure to take a scheduled dose or treatment can be problematic, or a threat to health or even a threat to life. To overcome the problem, the palatability enhancers of the instant invention can be administered in conjunction with the dietary supplement or medicament. In some embodiments, the palatability enhancer is provided as a coating or other topical application. In other embodiments, the palatability enhancer is admixed with the bulk of the dietary supplement or medicament. The composition of the palatability enhancer can be adapted, taking into account the aroma, taste and other properties of the dietary supplement of medicament, including any pronounced undesirable qualities (e.g., off-flavors, aromas), as well as the palatability preferences or likely palatability preferences of the animal for whom the product is intended. Preferably, the surfactant is a non-ionic surfactant.

In another aspect, the invention provides methods for enhancing the palatability of a comestible composition such as a food composition, dietary supplement, medicament, or other edible material. The methods comprise combining the comestible composition with an effective amount of a palatability enhancer comprising at least one animal digest and at least one surfactant. The animal digest and surfactant in the palatability enhancer are present in amounts of from about 0.05% to about 10% surfactant and from about 90% to about 99.95% animal digest. In various embodiments, the surfactant comprises from about 0.05% to 5% of the palatability enhancer, preferably from about 0.1% to 3% and more preferably from about 0.25% to about 1%, with the remainder being animal digest and other optional ingredients. The comestible composition, depending on the particular composition, comprises from about 0.001% to about 90% palatability enhancer, preferably from about 0.05% to 50%, more preferably from about 0.1% to 30%, and most preferably from about 1% to about 10%.

The palatability enhancer can be combined in any number of ways with the comestible composition before, during, or after the primary manufacturing step. In one embodiment, the combining step comprises topical application of the palatability enhancer on the primarily manufactured comestible composition. Topical application can be achieved by various ways known in the art, including spraying, enrobing, dipping, and liquid, dry or powder coating, or the like. In some embodiments, coatings or other surface or topical treatments are preferred because of the ease of application, and the versatility of applications for comestible compositions such as food compositions, dietary supplements, or medicaments. Further, the proximity of a coating or surface application is advantageous, in some embodiments, for maximizing the palatability enhancing properties of the animal digest and the surfactant. Rather than mixing in the bulk phase of the comestible composition, the palatability enhancer is concentrated on the surface where it can maximally interact with the animal's sensory systems, such as taste buds, olfactory tissues, and the like. Preferably, the surfactant is a non-ionic surfactant.

In one embodiment, the method further comprises an extrusion step. In various embodiments, the extrusion step occurs before, during, or at the time of the combining step. In certain embodiments, the extrusion step is used with a food composition or dietary supplement. For example, a food can be extruded, and the extruded food can later be combined with the palatability enhancer, such as in the form of a coating or surface application.

In another aspect, the invention provides methods for producing palatability enhancers. The methods generally comprise the step of combining at least one animal digest and at least one surfactant in an amount effective for enhancing the palatability of a comestible composition such as a food composition, dietary supplement, medicament, or other edible material. In one embodiment, the methods comprise a further step of emulsifying the animal digest and the surfactant. The emulsifying step can employ any technique known in the art for making emulsions. The palatability enhancers can be produced and used as is, or they can be further dried to remove any excess moisture for particular embodiments. The viscosity and solids concentration can be adjusted and standardized to allow for consistent coverage in coatings or other topical applications. The animal digest and surfactant are combined in amounts of from about 0.05% to about 10% surfactant and from about 90% to about 99.95% digest. In various embodiments, the surfactant comprises from about 0.05% to 5% of the palatability enhancer, preferably from about 0.1% to 3% and more preferably from about 0.25% to about 1%, with the remainder being animal digest and other optional ingredients. Preferably, the surfactant is a non-ionic surfactant.

In a further aspect, the present invention provides kits suitable for enhancing the palatability of a comestible composition such as a food composition, dietary supplement, medicament, or other edible material. The kits comprise in separate containers in a single package or in a virtual package, as appropriate for the kit component, a palatability enhancer comprising at least one animal digest and at least one surfactant in an amount effective for enhancing the palatability of a comestible composition and at least one of (1) one or more edible food ingredients; (2) a means for communicating information about or instructions for using the palatability enhancer for enhancing palatability; (3) a means for admixing the palatability enhancer with a comestible composition; (4) one or more dietary supplements; and (5) a medicament. The means for admixing the palatability enhancer with a comestible composition can be a simple container or mixing utensil suitable for mixing the palatability enhancer with a food composition, dietary supplement, or medicament. The means for admixing can comprise a device suitable of applying or coating the palatability enhancer onto a food composition, dietary supplement, or medicament, e.g., a spray bottle, atomizer, or other low-pressure applicator for applying a powdered, dry, moist, or liquid formulation of the palatability enhancer onto the surface of a comestible composition.

In another embodiment, the kits further comprise one or more agents, additional compositions, or medicaments for enhancing palatability or for improving appetite in an animal and further instructions for using the agents, additional compositions, or medicaments in conjunction with the palatability enhancer.

In another aspect, the invention provides a means for communicating information about, or instruction for use of, a palatability enhancer comprising at least one animal digest and at least one surfactant, e.g., a palatability enhancer as described herein. The information is about, or the instructions are for, one or more of (1) instructions for administering the palatability enhancer to an animal in conjunction with a food composition, dietary supplement, medicament, or other edible material; (2) instructions for one or more methods of using the palatability enhancer for enhancing the palatability of a food composition, dietary supplement, medicament, or other edible material; (3) information on providing proper nutrition, including the use of the composition, to an animal in need of foods having enhanced palatability, or an animal having a diminished appetite due to a disease or other health condition; (4) information about palatability, and appetite; (5) information regarding physical, cellular, and biochemical results of under nutrition, conditions causing loss of appetite, or wasting diseases, or recovery from, or prevention or treatment of the same; (6) comparative information or test results regarding the palatability enhancer; or (7) using the kits of the invention to enhance palatability of a comestible composition. The means of communicating comprises a physical or electronic document, digital storage media, optical storage media, audio presentation, audiovisual display, or visual display containing the information or instructions.

In various preferred embodiments, the means comprises any of a displayed web site, visual display kiosk, brochure, product label, package insert, advertisement, handout, public announcement, audiotape, videotape, DVD, CD-ROM, computer readable chip, computer readable card, computer readable disk, USB device, FireWire device, computer memory, and any combination thereof.

In another aspect, the invention provides packages comprising a palatability enhancer comprising at least one animal digest and at least one surfactant such as a non-ionic surfactant. The packages contain a word or words, picture, design, logo, graphic, symbol, acronym, slogan, phrase, or other device, or combination thereof, either directly on the package or on a label affixed thereto, indicating that the palatability enhancer is useful for enhancing the palatability of a comestible composition such as a food composition, dietary supplement, medicament, or other edible material.

In one embodiment, the package comprises a palatability enhancer of the present invention and a label affixed to the package containing a word or words, picture, design, acronym, slogan, phrase, or other device, or combination thereof, that indicates that the contents of the package contains a palatability enhancer with “enhanced palatability,” or that “may stimulate appetite,” “result in increased consumption,” “helps overcome loss of appetite,” “preferred palatability formula” or an equivalent expression printed on the package. In another embodiment, the package comprises a comestible composition such as food compositions, dietary supplements, medicaments, or other edible material having enhanced palatability. The comestible compositions comprise one or more ingredients suitable for consumption by an animal and at least one palatability enhancer comprising at least one animal digest and at least one surfactant in an amount effective for enhancing the palatability of the comestible composition. Any package or packaging material suitable for containing the composition is useful in the invention, e.g., a bag, box, bottle, can, pouch, or the like, manufactured from paper, plastic, foil, metal, and the like. In a preferred embodiment, the package contains a food composition adapted for a particular animal such as a human, canine or feline, as appropriate for the label, preferably a companion animal food composition.

### EXAMPLES

The invention can be further illustrated by the following example, although it will be understood that this example is included merely for purposes of illustration and is not intended to limit the scope of the invention unless otherwise specifically indicated.

### Example 1

Food compositions containing different amounts of compositions designed to increase palatability were tested to determine the relative palatability. To make the palatability enhancing compositions, the non-ionic surfactant Tween 80 was mixed with animal digest (poultry viscera hydrolysates) in the amounts shown in Table 1. The control used a composition that did not contain any Tween. Each palatability enhancing composition was applied to a separate batch of dry dog food composition by spray coating. The compositions were applied to produce food compositions containing approximately 0.25% and 0.5% of the palatability enhancing composition.

The food compositions were fed to panels of dogs and their preference for food observed and calculated. The animals were tested in panels of ˜20 dogs. The animals tested were similar in breed, age, sex, body weight, and body composition. The breeds used were: Big dogs (German Shepherd, Labrador Retriever); Medium dogs (Brittany Spaniel, Beagle, Fox Terrier); and Small dogs (Cavalier King Charles, Cairn Terrier). The tests were conducted in indoor kennel runs. The temperature was maintained at least at 15° C. Food compositions were provided ad libitum during 30 minutes. Food intake was sufficient to cover the dog's nutritional needs. Tap water was also provided ad libitum.

The experiment was design using a paired comparison test wherein two different food compositions were presented in parallel to each animal. A two-bowl system (bowls “A” and “B”) was used wherein one composition was presented in bowl “A,” and the other in bowl “B.”

Total Food Intake (g) for each dog was calculated by the difference: Wᵢₙ−Wₒᵤₜ, where Wᵢₙ is the total weight of food provided and Wₒᵤₜ is the total weight of food remaining (not consumed). Measurements were also made to determine the intake (g) individually of each of the compositions in bowls A and B, for each dog. Preference for A versus B was reflected in the calculated percentage consumption of composition A, calculated from the overall food intake as follows: % A=A/(A+B), where is A is the weight (g) of composition A consumed, and B is the weight of composition B consumed. A paired-t test was used to statistically compare results between the actual consumption of composition A relative to B (i.e., % A) and the “no preference” (i.e., 50%) or random level. The results are show in Table 1.

Referring to Table 1, the results show that food compositions containing non-ionic surfactants are more preferred (and thus, more palatable) than the control composition without the surfactant. The dogs in the tests preferred surfactant-containing dry dog food composition by a factor of 3:1 over control dry dog food compositions containing no surfactant.

TABLE 1

| | | | | | % of | | |
|---|---|---|---|---|---|---|---|
| | | | Grams | | Overall | | Number |
| | Bowl | Description | Con- | | Food | p | of |
| Variable | Side | of Variable | sumed | | Consumed | Value | Dogs |
| 8482.02 | A | Tween 80 | 237.8 | g | 77.50% | 0 | 19 |
| | | 0.5% in | | | | | |
| | | Digest | | | | | |
| 8482.01 | B | Control w/o | 69.7 | g | 22.50% | | |
| | | Tween 80 | | | | | |
| 8482.03 | B | Tween 80 | 250.6 | g | 74.30% | 0 | 19 |
| | | 0.25% in | | | | | |
| | | Digest | | | | | |
| 8482.01 | A | Control w/o | 86.5 | g | 25.70% | | |

Tween 80

### Example 2

A dietary supplement for an animal, preferably a companion animal, is produced, preferably in the form of a pill or caplet, or liquid formulation. One or more palatability enhancing compositions as described herein are produced, and added to the dietary supplement as a coating at from about 0.05% to about 5%, or added to a liquid or other formulation at from about 0.05-5% of the total composition. The supplement is provided to one or more animals in a side-by-side comparison with a control dietary supplement formulated without any palatability enhancing composition. The results show that the animals have a clear preference for the dietary supplement that includes the palatability enhancer(s).

### Example 3

A medicament for an animal, preferably a companion animal, is produced, preferably in the form of a pill or caplet, or liquid formulation. One or more palatability enhancing compositions as described herein are produced, and added to the medicament as a coating at from about 0.05% to about 5%, or added to a liquid or other formulation at from about 0.05-5% of the total composition. The medicament is provided to one or more animals in a controlled experiment(s) and the preference for the medicament relative to a control medicament formulated without any palatability enhancing composition. The results show that the animals have a clear preference for the medicament that is coated with, or includes the palatability enhancer(s).

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

1. A palatability enhancer comprising at least one animal digest and at least one surfactant in an amount effective for enhancing the palatability of a comestible composition.

2. The palatability enhancer of claim 1 comprising at least one non-ionic surfactant.

3. The palatability enhancer of claim 2 wherein the non-ionic surfactant comprises an alkyl poly(ethylene oxide); a copolymer of poly(ethylene oxide) and polypropylene oxide); an alkyl glucoside or polyglucoside; a fatty alcohol; a cocamide mono-, di-, or tri-ethanolamine, a lecithin derivative, a propylene glycol fatty acid ester, a glyceryl fatty acid ester, a polyglyceryl fatty acid ester, a polyoxyethylene glyceryl fatty acid ester, a sorbitan fatty acid ester, a polyoxyethylene sorbitan fatty acid ester, a polyoxyethylene sorbitol fatty acid ester, a polyethylene glycol fatty acid ester, a polyoxyethylene castor oil or hydrogenated castor oil, a polyoxyethylene alkyl ether, a polyoxyethylene phytosterol, a polyoxyethylene polyoxypropylene alkyl ether, or a polyoxyethylene lanolin, lanolin alcohol, or beeswax derivative.

4. The palatability enhancer of claim 1 comprising a non-ionic polyoxyethylene sorbitan fatty acid ester.

5. The palatability enhancer of claim 4 wherein the non-ionic surfactant is a polyoxyethylene sorbitan fatty acid monooleate.

6. The palatability enhancer of claim 1 wherein the animal digest comprises beef, pork, poultry, lamb, fish, or any combination thereof.

7. The palatability enhancer of claim 1 comprising from about 0.05% to 10% surfactant and from about 90% to about 99.95% animal digest.

8. The palatability enhancer of claim 1 further comprising one or more of a food-compatible acid or acidifier, a sweetening agent, an edible fat, an amino acid or its derivative or salt thereof, a salt, a nucleotide or ribonucleotide or its derivative or salt thereof.

9. The palatability enhancer of claim 8 wherein the food-compatible acid or acidifier comprises one or more of formic acid, acetic acid, citric acid, propionic acid, lactic acid, malic acid, fumaric acid, capric acid, caproic acid, butyric acid, hexamic acid, tartaric acid, gluconic acid, glutamic acid, glucuronic acid, guanylic acid, inosinic acid, kojic acid, itaconic acid, phosphoric acid, sodium bisulfate, pyrophosphoric acid, acid tripolyphosphate, acid hexapolyphosphate, and salts and derivatives thereof, and combinations of the foregoing.

10. A comestible composition comprising one or more ingredients suitable for consumption by an animal and at least one palatability enhancer comprising at least one animal digest and at least one surfactant in an amount effective for enhancing the palatability of the comestible composition.

11. The composition of claim 10 wherein the palatability enhancer comprises at least one non-ionic surfactant.

12. The composition of claim 10 wherein the animal digest comprises beef, pork, poultry, lamb, fish, or any combination thereof.

13. The composition of claim 10 wherein the palatability enhancer comprises from about 0.05% to 10% surfactant and from about 90% to about 99.95% animal digest.

14. The composition of claim 10 wherein the comestible composition comprises from about 0.001% to about 90% palatability enhancer.

15. The composition of claim 10 wherein the comestible composition is a food composition, dietary supplement, or medicament.

16. A method for enhancing the palatability of a comestible composition comprising combining the comestible composition with an effective amount of a palatability enhancer comprising at least one animal digest and at least one surfactant.

17. The method of claim 16 wherein the surfactant is a non-ionic surfactant.

18. The method of claim 16 further comprising an extrusion step wherein the extrusion step occurs before, during, or at the time of the combining step.

19. The method of claim 16 wherein the combining step comprises topical application of the palatability enhancer.

20. The method of claim 19 wherein the topical application comprises spraying or powder coating.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)
